(19) United States
(12) Patent Application Publication
(10) Pub. No.: US 2024/0167997 A1
(43) Pub. Date: May 23, 2024

(54) Simulating Dissolution of Scale in Wells

(71) Applicant: Saudi Arabian Oil Company, Dhahran (SA)

(72) Inventors: Mohammed Sayed, Katy, TX (US); Fakuen Frank Chang, Houston, TX (US); Amy J. Cairns, Houston, TX (US); Tim Luce, Houston, TX (US)

(21) Appl. No.: 17/991,076

(22) Filed: Nov. 21, 2022

(51) Int. Cl. G01N 33/24; G01N 21/73; G01N 23/046; G01N 23/083

(52) U.S. Cl. CPC G01N 33/24; G01N 21/73; G01N 23/046; G01N 23/083; G01N 2223/04; G01N 2223/419; G01N 2223/616

(57) ABSTRACT

Provided are systems and methods related to a laboratory core flooding device that simulates inflow control devices (ICD) or valves (ICV) geometry in a well completion.

### TECHNICAL FIELD

This specification describes systems and methods related to a laboratory core flooding apparatus that simulates or mimics inflow control devices (ICD) or valves (ICV) geometry in a well completion.

### BACKGROUND

Subterranean hydrocarbon (e.g., oil, gas, condensate) bearing formations may be mechanically weak when they contain reef, lagoonal, and/or deep-water carbonate accumulations. Such formations can contain calcite (e.g., 90-100 wt. %), as well as dolomite (e.g., 0-8 wt. %), ankerite, pyrite, siderite, gypsum, halite, sylvite and/or barite as minor minerals. Well completion strategies for such formations sometimes include a long horizontal lateral exposed to rock with variable permeability and formation sections with different pressures. ICDs and/or valves ICVs can be installed along the lateral to even the flow profile along the well drainage area.

During the drilling and well completion process, carbonate particulates, a weighting agent component of the drilling fluid, may deposit on the ICDs and/or ICVs. During production, carbonate scale may precipitate and/or reservoir solids may migrate into the ICDs and/or ICVs causing the valve stem to get stuck on the seats, preventing proper opening and closing. Repairing stuck ICDs and ICVs with a chemical suitable to remove the scale and particulate deposit without triggering additional dissolution of the carbonate formation rock is difficult.

### SUMMARY

This specification describes systems and methods for simulating ICD or ICV geometry in a well completion. A system for simulating invasion of a reactive fluid for scale removal in a formation matrix includes a laboratory core flooding apparatus that uses two rock samples: one representing the solid scale deposits on the ICD/ICV and one representing the formation matrix. A treatment fluid is injected through a first section of the device and can then interact with the carbonate sample representing scale. The quantity of treatment fluid that invades the formation matrix after it contacts the scale in the ICD/ICV can be assessed using the lab device.

In a first aspect, the disclosure provides a method of simulating invasion of a fluid for treating scale into a formation matrix. The method includes forming a test sample by inserting a cylindrical sample representing the formation matrix into a sleeve sized to fit in a core holder of a core flood system, the sleeve having a first open end and a second open end; inserting a first cylindrical metal spacer into the sleeve, the first spacer defining a first central bore extending axially through the first cylindrical metal spacer; inserting a cylindrical sample representing scale into the sleeve; inserting a second cylindrical metal spacer into the sleeve, the second spacer defining a second central bore extending axially through the second cylindrical metal spacer, wherein the second central bore has a larger diameter than the first central bore; such that the cylindrical sample representing the formation matrix is between the first open end of the sleeve and the first cylindrical metal spacer, the first cylindrical metal spacer is between the cylindrical sample representing the formation matrix and the cylindrical sample representing scale, and the second cylindrical metal spacer is between the cylindrical sample representing scale and the second open end of the sleeve; inserting test sample into the core holder of the core flood system; injecting the fluid for treating scale into the second central bore of the test sample in the core holder; aging the test sample; measuring at least one parameter selected from the group consisting of a pressure of the core flood system, a temperature of the core flood system, and a flow rate of the fluid; and injecting water into the core holder in a direction reverse to a flow of the fluid.

In some embodiments, the test sample is formed with the cylindrical sample representing the formation matrix adjacent the first cylindrical metal spacer, the first cylindrical metal spacer adjacent the cylindrical sample representing scale, and cylindrical sample representing scale adjacent the second cylindrical metal spacer.

In some embodiments, the test sample is formed with the cylindrical sample representing the formation matrix adjacent the first open end of the sleeve and the second cylindrical metal spacer adjacent the second open end of the sleeve.

In some embodiments, the cylindrical sample representing scale includes marble.

In some embodiments, the cylindrical sample representing the formation matrix includes a core taken from a subsurface formation.

In some embodiments, the fluid includes an acid.

In some embodiments, injecting the fluid includes injecting 0.1 to 10 times a volume of fluid calculated to completely dissolve the cylindrical sample representing scale.

In some embodiments, the method further includes applying a temperature of 150° F. to 350° F., an overburden pressure of 2000 to 4000 psi, and a backpressure of 500 to 3000 psi to the test sample while aging the test sample, wherein the backpressure is at least 1000 psi less than the overburden pressure.

In some embodiments, the method further includes collecting the fluid and the injected water and analyzing the fluid and the injected water by ICP.

In some embodiments, the method further includes measuring the cylindrical sample representing the formation matrix and the cylindrical sample representing scale using X-ray tomography.

In a second aspect, the disclosure provides a system for simulating invasion of a fluid for treating scale into a formation matrix. The system includes a rubber sleeve defining an interior space; a first cylindrical rock sample disposed in the interior space of the rubber sleeve; a second cylindrical rock sample disposed in the interior space of the rubber sleeve; a first cylindrical metal spacer disposed in the interior space of the rubber sleeve between the first cylindrical rock sample and the second cylindrical rock sample, the first spacer defining a first central bore extending axially through the first cylindrical metal spacer; and a second cylindrical metal spacer disposed in the interior space of the rubber sleeve between the second cylindrical rock sample and an open end of the interior space, the second cylindrical metal spacer defining a second central bore extending axially through the second cylindrical metal spacer. An outer diameter of the first cylindrical metal spacer and an outer diameter of the second cylindrical metal spacer abut an inner diameter of the rubber sleeve. The second central bore has a larger diameter than the first central bore. The rubber sleeve defines a length greater than a sum of lengths of the first cylindrical metal spacer, the second cylindrical metal spacer, the first cylindrical rock sample, and the second cylindrical rock sample.

In certain embodiments, the first cylindrical metal spacer is in contact with the first cylindrical rock sample and the second cylindrical rock sample; and the second cylindrical metal spacer is in contact with the second cylindrical rock sample.

In certain embodiments, the system further includes a core flood system and the rubber sleeve is sized to fit in a sample chamber of the core flood system.

In certain embodiments, the system further includes an acid; and the core flood system delivers the acid to the central bore of the second cylindrical metal spacer.

In a third aspect, the disclosure provides a system for simulating invasion of a fluid for treating scale into a formation matrix. The system includes a rubber sleeve having an inner diameter defining an interior space; a first cylindrical metal spacer defining a first central bore extending axially through the first cylindrical metal spacer, the first cylindrical metal spacer having an outer diameter sized to abut the inner diameter of the rubber sleeve; and a second cylindrical metal spacer defining a second central bore extending axially through the second cylindrical metal spacer, the second cylindrical metal spacer having an outer diameter sized to abut the inner diameter of the rubber sleeve. The second central bore has a larger diameter than the first central bore. The rubber sleeve has a length at least 0.5 inches longer than a combined length of the first cylindrical metal spacer and the second cylindrical metal spacer.

In certain embodiments, the first cylindrical metal spacer and the second metal spacer have an outer diameter of 1 inch to3 inches.

In certain embodiments, the first central bore of the first cylindrical metal spacer has a diameter of 0.25 inches to1.19 inches.

In certain embodiments, the second central bore of the second cylindrical metal spacer has a diameter of 0.25 inches to1.19 inches.

In certain embodiments, the first cylindrical metal spacer has a length of 0.5 inches to3 inches.

In certain embodiments, the second cylindrical metal spacer has a length of 0.5 inches to3 inches.

In certain embodiments, the rubber sleeve has a length of 6 inches to24 inches.

The systems and methods of the disclosure can enable the simulation of scale formed during a well completion and the formation around the well components. The systems and methods of the disclosure enable the evaluation of the performance of different acid formulations for scale dissolution and penetration to the formation as well as implementation procedures.

In some approaches, the completion system is removed from the well for cleaning using suitable chemical or mechanical means, followed by reinstallation into the well, to avoid dissolving the rock formation. The systems and methods of this disclosure enable the ICD to function properly without removal of the completion system whereby a suitable treatment fluid is injected in situ downhole to remove the scale deposits. This approach reduces operational complexity as well as cost and time associated with removal and reinstallation of the completion system.

The details of one or more embodiments of these systems and methods are set forth in the accompanying drawings and the description below. Other features, objects, and advantages of these systems and methods will be apparent from the description and drawings, and from the claims.

### DESCRIPTION OF DRAWINGS

FIG. 1 is a schematic illustration of a core flooding system.

FIG. 2 is a schematic illustration of system for simulating dissolution of scale deposits in wells.

FIGS. 3A and 3B are photographs of the system for simulating dissolution of scale deposits in wells.

FIG. 4 is a flow chart of a method of using a core flooding system.

FIG. 5 is a graph of pressure over time.

FIG. 6 is a graph of the metal ion concentrations as a function of time.

FIG. 7 is a photograph of first and second cylindrical outcrop rock samples.

FIG. 8 presents X-Ray topographies of first and second cylindrical rock samples.

FIG. 9 is a graph of pressure as a function of time.

FIG. 10 is a photograph of first and second cylindrical outcrop rock samples.

FIG. 11 presents X-Ray topographies of first and second cylindrical outcrop rock samples.

FIGS. 12A and 12B are photographs of first and second cylindrical outcrop rock samples.

FIG. 13 presents X-Ray topographies of first and second cylindrical outcrop rock samples.

Like reference symbols in the various drawings indicate like elements.

### DETAILED DESCRIPTION

Stimulation in carbonate reservoirs is often conducted using hydrochloric acid (HCl) where large volumes of acid are injected to dissolve the rock matrix thereby creating highly conductive flow channels that enable hydrocarbon recovery. Wellbore stability after drilling, and rock strength are a concern in weak formations. Use of highly reactive acid treatments in such scenarios can cause well enlargement and borehole instability, risking wellbore collapse and/or solid production. A treatment designed to dissolve or mobilize the scale thereby allowing the ICD to function properly without negatively impacting the mechanical integrity of the formation matrix is important to maintain well productivity.

This specification describes systems and methods related to a laboratory core flooding device that simulates ICD or ICV geometry in a well completion. The systems simulate the deposit of scale on ICDs and the presence of the soft formation matrix that should not be contacted with acids. The system includes a rock sample that represents the scale deposited on the ICDs and valves and a rock sample that represents the soft reservoir formation. Between the two rock samples, a first spacer with a relatively small diameter hole is present to simulate a nick of the ICD's seating that can open and close to allow production from the well. A second spacer with a relatively large radius hole is present in front of the rock representing the scale deposited. The second spacer is used to simulate the space available for the stimulation fluid to contact the scale formed on the valves and/or ICD stem/seat. The setup is designed such that a volume of acid sufficient to fill the hole of the second spacer can be injected, then the acid contacts the rock sample representing the scale deposited. The rock sample representing scale deposited reacts with the acid upon contact. It is desirable that the acid preferentially dissolves the scale formation, thereby becoming spent acid, without interacting with the rock sample representing the soft reservoir. However, the acid may react with the scale and penetrate through the first spacer (representing the ICD stem) and react with the soft formation sample. Different acid solutions can be assessed to determine the degree of penetration to the soft formation that may take place during a downhole completion.

Table 1 shows a typical composition of collected debris from a well using X-ray powder diffraction (PXRD) analysis after acid digestion and washing with water. The debris material consisted mainly of calcite (CaCO₃) (88 wt. %) with minor amounts of quartz (SiO₂), dolomite (CaMg(CO₃)₂), albite (NaAlSi₃O₈), and barite (BaSO₄). Table 1 shows that the calcite and dolomite can be dissolved by acid.

TABLE 1
Estimated mineral composition (wt %) of recovered
solid debris (i.e. scale deposits) as received
from a well and percentage dissolved in HCl

| | | After Dissolved in |
|---|---|---|
| Mineral, wt. % | Received Debris | 10 wt. % HCl |
| Calcite (CaCO3) | 88 | 0 |
| Dolomite [CaMg(CO3)2] | 1 | 0 |
| Quartz (SiO2) | 9 | 96 |
| Barite (BaSO4) | 1 | 2 |
| Albite (NaAlSi3O8) | 1 | 2 |

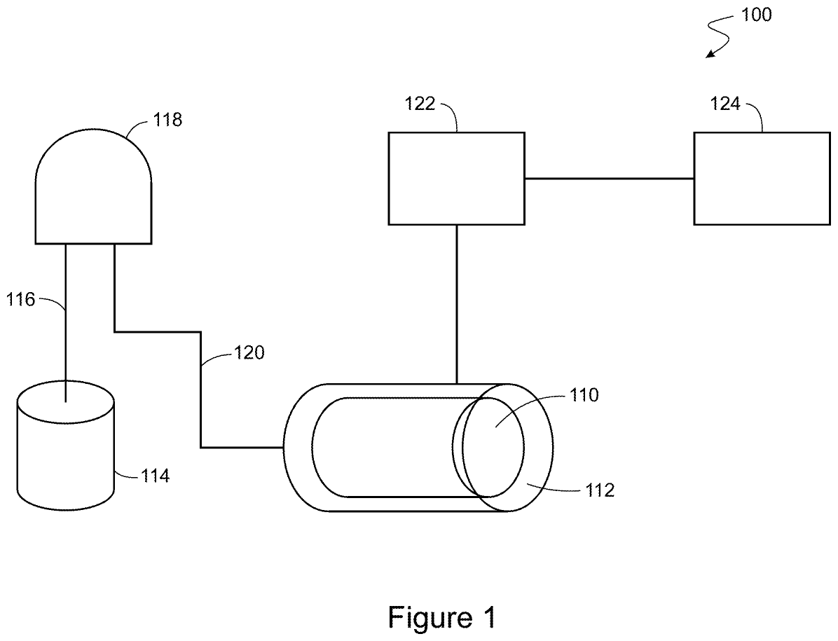

FIG. 1 depicts a core flood system 100 that can be used to conduct various tests (e.g., permeability tests, formation damage tests, stimulation of reservoir matrix carbonates or sandstones, enhanced oil recovery experiments and testing, regain permeability measurements after fracturing fluids, sand consolidation testing, testing clay stabilizers, condensate recovery and condensate banking mitigation). Core floods are usually performed at a given temperature under the conditions of constant pump flow pressure across the core plugs or constant flow through the core plugs. Core flood tests can be conducted using single core holders or multiple core holders run in series or parallel.

The core flood system 100 includes a sample 110, a core holder 112, an acid solution 114, a first conduit 116, a pump 118, a second conduit 120, a pressure sensor 122, and a computer 124. The sample 110 is disposed inside the core holder 112. The acid solution 114 is connected to the pump 118 through a first conduit 116. The pump 118 delivers the acid solution 114 to the core holder 112 containing the sample 110 through the second conduit 120. The pressure sensor 122 senses the pressure inside the core holder 112 and the data is sent to the computer 124.

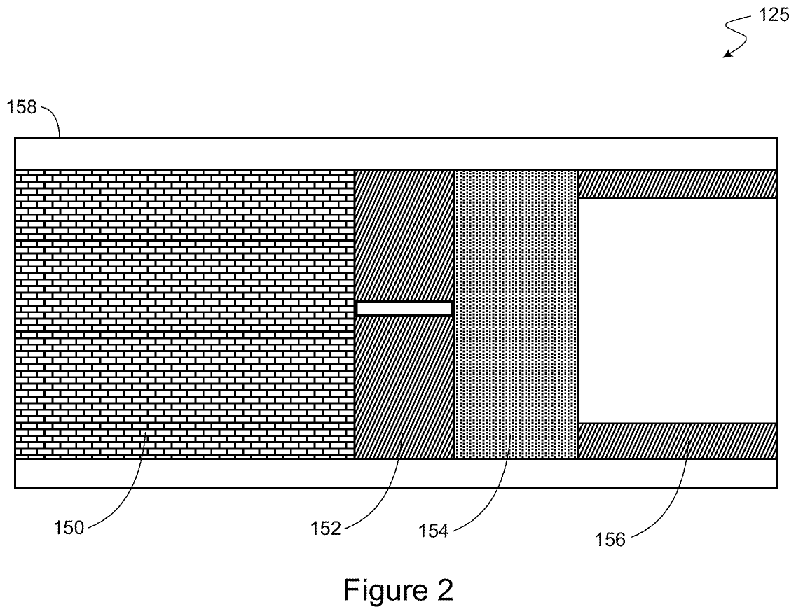

FIG. 2 depicts a system 125 that is used to simulate scale on ICDs or ICVs and the soft formation. The system 125 contains a first rock sample 150, a first spacer 152, a second rock sample 154, a second spacer 156 and a rubber sleeve 158. The first rock sample 150, the first spacer 152, the second rock sample 154, and the second spacer 156 are disposed in the rubber sleeve 158. The first spacer 152 has a small diameter bore to represent the valve seat that is controlling flow to the formation. The second spacer 156 has a large diameter bore that can contain the acid solution 114. The bores of the first spacer 152 and the second spacer 156 are located at or near the center of the first spacer 152 and the second spacer 156, respectively. The diameter of the spacer is selected based on the diameter of the available core samples. The core samples are drilled in diameters of 1 and 1.5 inches. The diameter of the bore of the first spacer 152 matches the internal diameters of the core flood system tubing. The diameter of the bore of the second spacer 156 is selected to provide a sufficient volume of acid for the reaction to take place. The first spacer 152 can have a length of 1 inch, an outer diameter of 1.5 inches and a bore diameter of 0.25 inches to1.19 inches (3 cm). The second spacer 156 can have a length of 1 inch, an outer diameter of 1.5 inches and a bore diameter of 0.55 inches (1.4 cm). The first rock sample 150, representing the reservoir, and the second rock sample, representing the scale, are sized to fit snugly within the rubber sleeve 158. A shorter length is desirable for the first rock sample 150 representing scale to allow for the acid to breakthrough and invade reservoir sample. For the second rock sample 154 representing the reservoir, the sample should be sufficiently long to enable the detection of wormhole propagation under X-ray tomography. Typically, the first rock sample has a diameter between 1 inch and 1.5 inches and a length between 0.5 inches and 3 inches. Typically, the second rock sample 154 has a diameter between 0.55 inches and 1.5 inches and a length between 0.5 inches and 3 inches. The rubber sleeve 158 has a diameter between 1 inch and 1.5 inches and a length between 6 and 20 inches. The system 125 can be used as the sample 110 in the core flood system 100.

During well completion and/or production, carbonate scale may precipitate and/or reservoir solids may migrate into the ICDs and/or ICVs causing the valve stem to get stuck on the seats, preventing them from opening and closing properly. The first rock sample 150 represents the formation matrix and the second rock sample 154 represents the scale and solid deposit on the ICD/ICV.

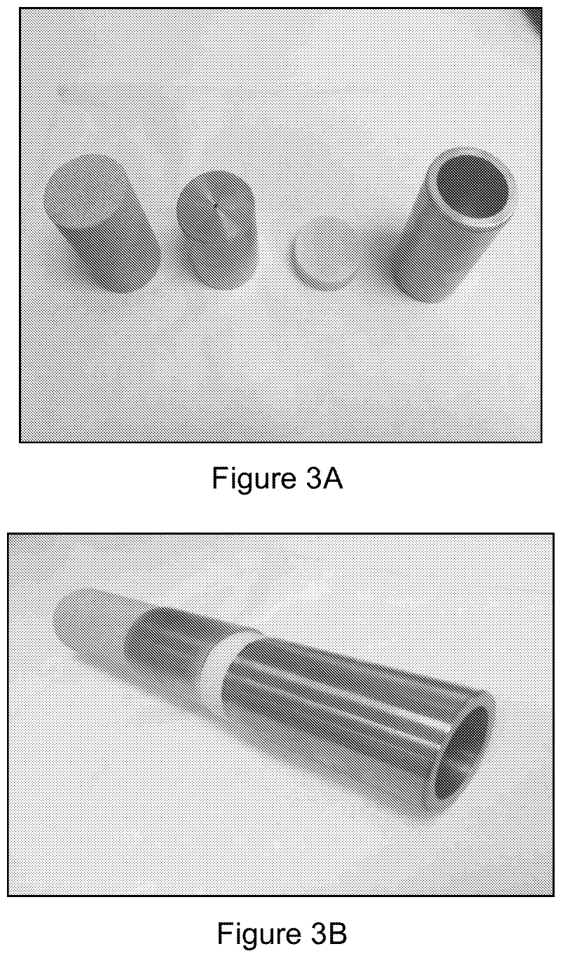

FIGS. 3A and 3B are photographs of a prototype of the system 125 without the rubber sleeve. From left to right, FIG. 3A shows an Indiana limestone core with a length of 3 inches used to represent the formation, a first spacer (1.0″ Length×1.500″ Diameter and 0.250″ bore), a Marble core sample with a length of 1 inch to represent the scale, and the second spacer (1.0″ Length×1.500″ Diameter and 1.18″ bore). FIG. 3B shows the components of FIG. 3A positioned as they would be when tested in the core flood system 100.

The system 125 is designed to simulate downhole conditions capable of operating under harsh conditions, i.e., up to temperatures of −100° F. and pressures of 10,000 psi. For example, the first spacer 152 and the second spacer 156 can be formed of an alloy resistant to corrosion (e.g., alloys containing nickel, molybdenum and chromium). In a prototype system, the first spacer 152 and the second spacer 156 (the wetted components) were formed of Hastelloy HC-276.

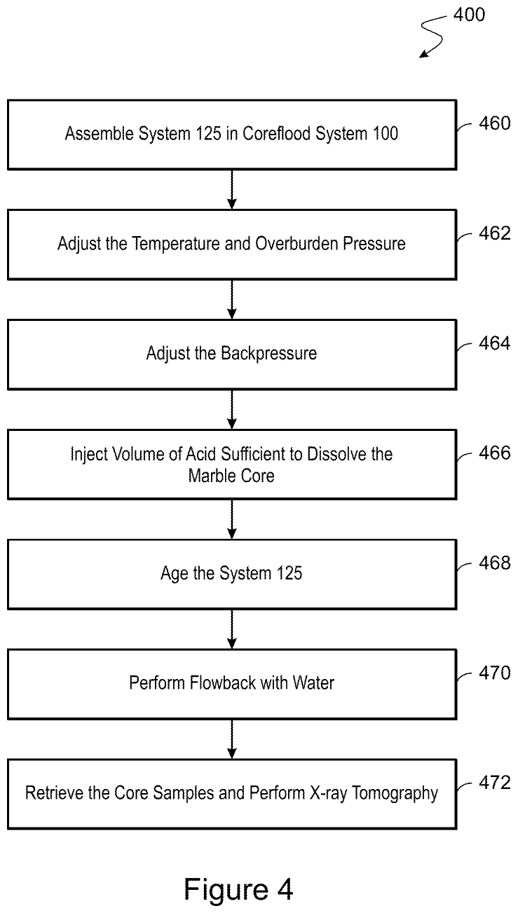

FIG. 4 shows a flowchart 400 of a method to test the system 125. In step 460, the system 125 is assembled as depicted in FIG. 2 and placed in the coreflood system 100. In step 462, the temperature and overburden pressure are set to simulate the reservoir temperature and pressure, typically 150 to 325° F. and 2000 to 3000 psi. In step 464, the backpressure is set, typically at a value of 1000 psi to control the generation of carbon dioxide (CO₂) and keep the majority of the CO₂ dissolved in solution. A value between 500 to 3000 psi can be used for the backpressure, however, the overburden pressure is maintained at a pressure least 1000 psi higher than the back pressure. In step 466, a volume of acid sufficient to dissolve the second rock sample 154 (representing scale), and fill the large hole of the second metal spacer 156 is injected. The volume of acid is calculated based on the mineral composition of the rock, type of acid and concentration of acid such that it will dissolve at least 50% of the first rock sample representing the scale. In step 468, the system 125 is aged, typically from 3 to 24 hours with 3 to 6 hours being common in acidizing jobs. During the aging, the pressure, temperature and flow rate are monitored over time. After aging the sample, in step 470, a flowback with water is performed. If the acid was able to break through the marble representing the scale, the flowback is continued. If breakthrough did not occur, the flowback is terminated and the remaining acid is collected. The water from the flowback and/or the remaining acid are measured using inductively coupled plasma (ICP) to determine cation concentrations. In step 472, the first rock sample 150 and the second rock sample 156 are collected and X-ray tomography (CT scan) is performed to detect the dissolution pattern and the formation of wormholes.

The prototype was tested with three different acid systems. During the tests, the system temperature was adjusted to190° F., the overburden pressure was adjusted to a value of 2000 psi, and the backpressure was adjusted to a value of about 500 psi.

For ICP measurements, aliquots of sample were collected and used to prepare dilution solutions in deionized water in ratios of 10, 100 and 1000 dilution. A six-point calibration curve that contains a 0.00 ppm calibration blank and standards at 1, 10, 25, 50 and 100 ppm was constructed and verified at concentrations of 0.00, 10 and 100 ppm (corresponding to calibration standards ICB, ICV1 and ICV2, respectively). The calibration was verified throughout the analysis every 25 sample readings. After the calibration or calibration verification standards, the samples are measured. ICP measurements were performed on a Perkin Elmer Inductively Coupled Plasma Optical Emission Spectrometer (ICP-OES).

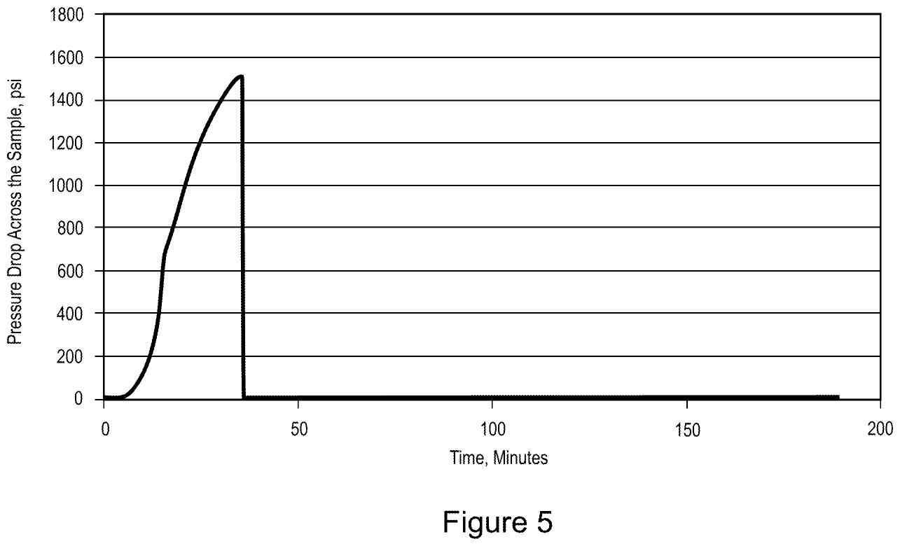

In a first test, 72 mL of 28 wt % hydrochloric acid (HCl) was injected the system to fill the large hole of the second spacer. The sample was aged for three hours while recording the sample pressure, temperature and flow rate over time. The measured pressure drop across the sample was plotted as a function of the time of injection in minutes and shown in FIG. 5. The pressure drop began to increase due to the generation and accumulation of CO₂ toward the inlet of the coreflood. The acid injected broke through the marble core, flowed through the space and reacted with the Indiana limestone outcrop core sample representing a subsurface formation of the reservoir. The reaction continued until acid reached the end of the core sample, referred to as acid breakthrough, as indicated by the sudden drop in differential pressure across the core. This breakthrough occurred during the aging step. Immediately, deionized water (DI-H₂O) was injected in the reverse direction and effluent samples were collected and the cation concentrations were measured by ICP analysis.

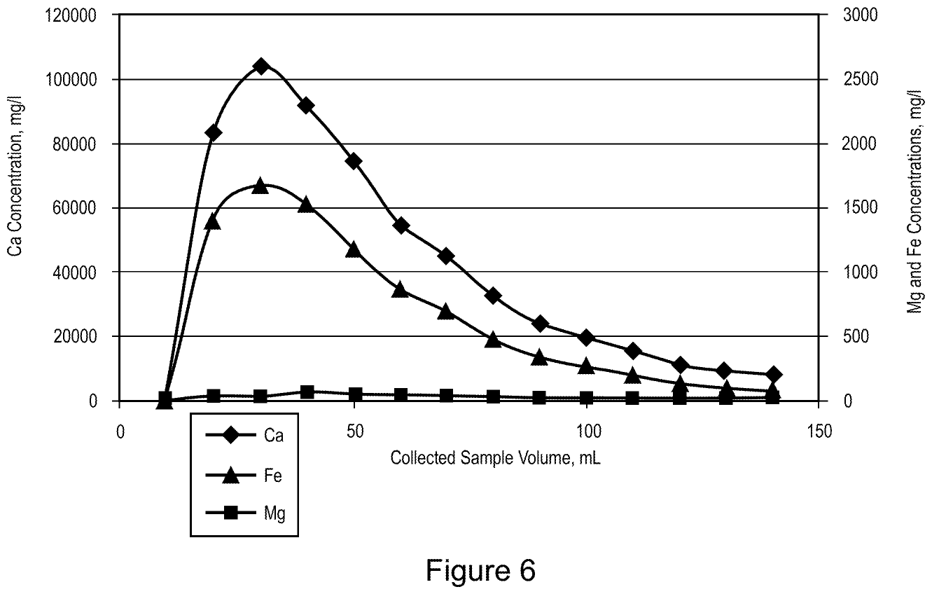

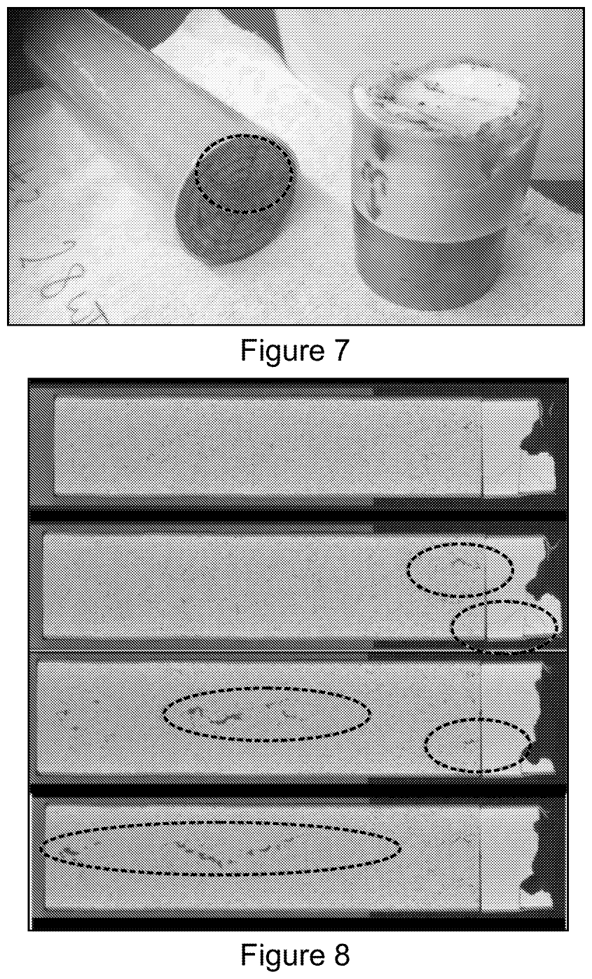

The results of the ICP analysis are shown in FIG. 6. Digital and X-ray tomography photos of the rock samples are shown in FIGS. 7 and 8. The 28 wt. % HCl aggressively reacted with the marble (scale representing core sample) and invaded the Indiana Limestone core (representing the reservoir rock) indicating that this acid system is not suitable for scale treatment as the scale cannot be dissolved without causing damage to the formation.

In a second test, 70 mL of a slow-reacting acid system referred to as LVAS-1 for Low Viscosity Acid System was injected into the system, followed by aging for three hours to allow the acid to react with the marble core sample. The composition of LVAS-1 is shown in Table 2.

TABLE 2
Composition of LVAS-1 Acid System

| Additive Description | Unit | Amount per 1000 gal |
|---|---|---|
| LV-002, Corrosion Inhibitor | Gal. | 20 |
| A-153, Inhibitor Intensifier | Gal. | 70 |
| LV-0001, Organic Acid | Lbs. | 487.5 |
| Methanesulfonic acid | | |
| L-058, Iron Control Agent | Lbs. | 4 |
| L-041, Chelating Agent | Gal. | 50 |
| H-031, 31% Hydrochloric Acid | Gal. | 487.5 |
| M-295 H₂S Scavenger | Gal. | 5 |

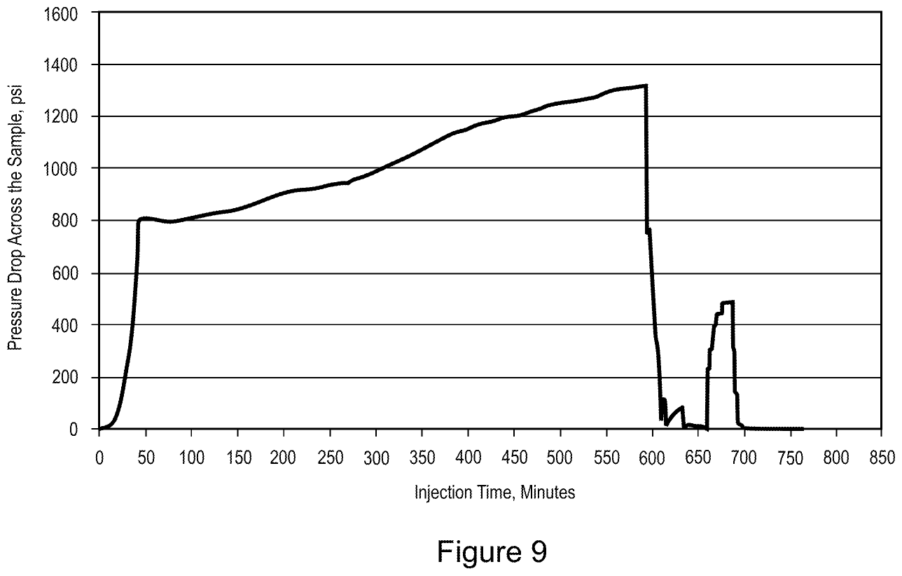

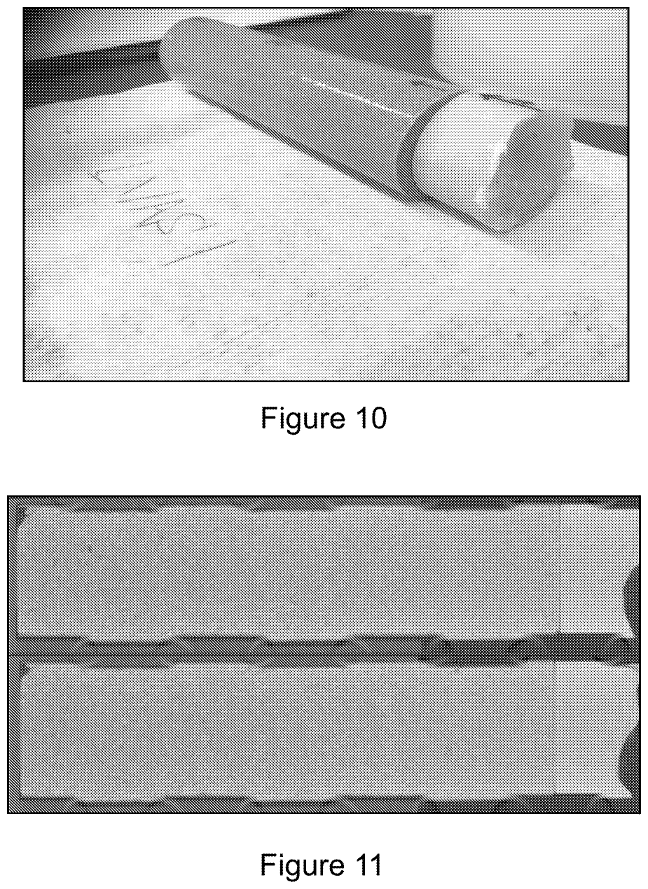

The collected pressure drop across the sample was plotted as a function of time (expressed as pore volume of the reservoir rock or the Indiana limestone) and is shown in FIG. 9. The pressure drop started to increase as a result of CO₂ generation and accumulation after 10 minutes of injection. The injected acid did not break through the marble core, or reach the reservoir core sample as there was no flow after the aging step. Most of the acid reacted with the marble core sample (representing scale deposited on the ICVs). Digital and X-ray tomography photos of the core samples are shown in FIGS. 10 and 11, respectively. LVAS-I aggressively reacted with the marble but it did not invade or react with the reservoir. The results indicate that LVAS-I is suitable for scale treatment, as scale can be dissolved without causing damage to the formation.

In a third test, 70 mL of a mineral acid/glycol blend denoted as Low Cost Acid System I (LCAS-I) was injected into the system, followed by aging for 3 hours to allow the acid to react with the marble core sample. The composition of LCAS-I is shown in Table 3.

TABLE 3
Composition of LCAS-I Acid System

| Component | Vol/100 mL of acid |
|---|---|
| 28 wt % HCl | 79 |
| Diethylene Glycol | 19 |
| F25 Corrosion Inhibitor | 2 |

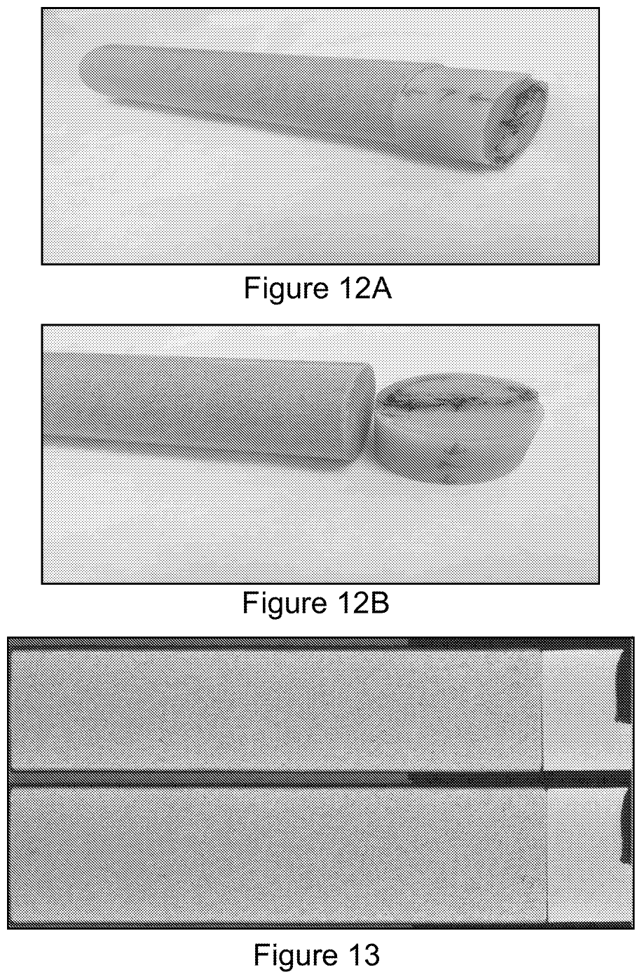

The collected pressure drop across the sample showed no evidence of breakthrough across the marble or the reservoir core sample. Most of the acid reaction took place with the marble core sample representing scale deposited on the ICVs. Digital and X-ray tomography photos are shown in FIGS. 12A and 12B and 13, respectively. LCAS-I, aggressively reacted with the marble but did not invade or react with the reservoir. The results show that LCAS-I can used for scale treatment, as scale can be dissolved without causing damage to the formation.

A number of embodiments of the invention have been described. Nevertheless, it will be understood that various modifications may be made without departing from the spirit and scope of the invention. For example, a third metal space can be employed where the third spacer is used as a sleeve for the second rock sample 154 to keep it between the first metal spacer 152 and the second metal spacer 156. This enables the use of smaller samples for the second rock sample 154. Accordingly, other embodiments are within the scope of the following claims.

What is claimed is:

1. A method of simulating invasion of a fluid for treating scale into a formation matrix, the method comprising:
forming a test sample by:
inserting a cylindrical sample representing the formation matrix into a sleeve sized to fit in a core holder of a core flood system, the sleeve having a first open end and a second open end;
inserting a first cylindrical metal spacer into the sleeve, the first spacer defining a first central bore extending axially through the first cylindrical metal spacer;
inserting a cylindrical sample representing scale into the sleeve;
inserting a second cylindrical metal spacer into the sleeve, the second spacer defining a second central bore extending axially through the second cylindrical metal spacer, wherein the second central bore has a larger diameter than the first central bore;
such that the cylindrical sample representing the formation matrix is between the first open end of the sleeve and the first cylindrical metal spacer, the first cylindrical metal spacer is between the cylindrical sample representing the formation matrix and the cylindrical sample representing scale, and the second cylindrical metal spacer is between the cylindrical sample representing scale and the second open end of the sleeve;
inserting test sample into the core holder of the core flood system;
injecting the fluid for treating scale into the second central bore of the test sample in the core holder;
aging the test sample;
measuring at least one parameter selected from the group consisting of a pressure of the core flood system, a temperature of the core flood system, and a flow rate of the fluid; and
injecting water into the core holder in a direction reverse to a flow of the fluid.

2. The method of claim 1, wherein the test sample is formed with the cylindrical sample representing the formation matrix adjacent the first cylindrical metal spacer, the first cylindrical metal spacer adjacent the cylindrical sample representing scale, and cylindrical sample representing scale adjacent the second cylindrical metal spacer.

3. The method of claim 2, wherein the test sample is formed with the cylindrical sample representing the formation matrix adjacent the first open end of the sleeve and the second cylindrical metal spacer adjacent the second open end of the sleeve.

4. The method of claim 1, wherein the cylindrical sample representing scale comprises marble.

5. The method of claim 4, wherein the cylindrical sample representing the formation matrix comprises a core taken from a subsurface formation.

6. The method of claim 1, wherein the fluid comprises an acid.

7. The method of claim 6, wherein injecting the fluid comprises injecting 0.1 to 10 times a volume of fluid calculated to completely dissolve the cylindrical sample representing scale.

8. The method of claim 1, further comprising applying a temperature of 150° F. to 350° F., an overburden pressure of 2000 to 4000 psi, and a backpressure of 500 to 3000 psi to the test sample while aging the test sample,
wherein the backpressure is at least 1000 psi less than the overburden pressure.

9. The method of claim 1, further comprising collecting the fluid and the injected water and analyzing the fluid and the injected water by ICP.

10. The method of claim 9, further comprising measuring the cylindrical sample representing the formation matrix and the cylindrical sample representing scale using X-ray tomography.

11. A system for simulating invasion of a fluid for treating scale into a formation matrix, the system comprising:
a rubber sleeve defining an interior space;
a first cylindrical rock sample disposed in the interior space of the rubber sleeve;
a second cylindrical rock sample disposed in the interior space of the rubber sleeve;
a first cylindrical metal spacer disposed in the interior space of the rubber sleeve between the first cylindrical rock sample and the second cylindrical rock sample, the first spacer defining a first central bore extending axially through the first cylindrical metal spacer;
a second cylindrical metal spacer disposed in the interior space of the rubber sleeve between the second cylindrical rock sample and an open end of the interior space, the second cylindrical metal spacer defining a second central bore extending axially through the second cylindrical metal spacer;
wherein an outer diameter of the first cylindrical metal spacer and an outer diameter of the second cylindrical metal spacer abut an inner diameter of the rubber sleeve;
wherein the second central bore has a larger diameter than the first central bore; and
wherein the rubber sleeve defines a length greater than a sum of lengths of the first cylindrical metal spacer, the second cylindrical metal spacer, the first cylindrical rock sample, and the second cylindrical rock sample.

12. The system of claim 11, wherein the first cylindrical metal spacer is in contact with the first cylindrical rock sample and the second cylindrical rock sample; and
the second cylindrical metal spacer is in contact with the second cylindrical rock sample.

13. The system of claim 11, wherein the system further comprises a core flood system and the rubber sleeve is sized to fit in a sample chamber of the core flood system.

14. The system of claim 13, wherein the system further comprises an acid; and the core flood system delivers the acid to the central bore of the second cylindrical metal spacer.

15. A system for simulating invasion of a fluid for treating scale into a formation matrix, the system comprising:
a rubber sleeve having an inner diameter defining an interior space;
a first cylindrical metal spacer defining a first central bore extending axially through the first cylindrical metal spacer, the first cylindrical metal spacer having an outer diameter sized to abut the inner diameter of the rubber sleeve;
a second cylindrical metal spacer defining a second central bore extending axially through the second cylindrical metal spacer, the second cylindrical metal spacer having an outer diameter sized to abut the inner diameter of the rubber sleeve;
wherein the second central bore has a larger diameter than the first central bore; and
wherein the rubber sleeve has a length at least 0.5 inches longer than a combined length of the first cylindrical metal spacer and the second cylindrical metal spacer.

16. The system of claim 15, wherein the first cylindrical metal spacer and the second metal spacer have an outer diameter of 1 inch to3 inches.

17. The system of claim 16, wherein the first central bore of the first cylindrical metal spacer has a diameter of 0.25 inches to1.19 inches.

18. The system of claim 17, wherein the second central bore of the second cylindrical metal spacer has a diameter of 0.25 inches to1.19 inches.

19. The system of claim 18, wherein the first cylindrical metal spacer has a length of 0.5 inches to3 inches.

20. The system of claim 19, wherein the second cylindrical metal spacer has a length of 0.5 inches to3 inches.

21. The system of claim 20, wherein the rubber sleeve has a length of 6 inches to 24 inches.
